(19) United States
(12) Patent Application Publication
(10) Pub. No.: US 2021/0291152 A1
(43) Pub. Date: Sep. 23, 2021

(54) PREPARATION OF NITROGEN RICH THREE DIMENSIONAL MESOPOROUS CARBON NITRIDE AND ITS SENSING AND PHOTOCATALYTIC PROPERTIES

(71) Applicant: SABIC Global Technologies B.V.

(72) Inventors: Gurudas P. Mane, Mawson Lakes (AU); Ajayan Vinu, Mason Lakes (AU); Ugo Ravon, Thuwal (SA); Khalid Al-Bahily, Thuwal (SA)

(21) Appl. No.: 16/324,942

(22) PCT Filed: Aug. 8, 2017
(86) PCT No.: PCT/IB2017/054847

§ 371 Date: Feb. 12, 2019

(51) Int. Cl. B01J 27/24; B01J 23/42; B01J 35/00; B01J 35/10; B01J 37/00; B01J 37/08; C01B 3/04; B01J 19/12; G01N 33/50; G01N 21/64

(52) U.S. Cl. CPC B01J 27/24; G01N 2201/06113; B01J 35/004; B01J 35/1019; B01J 35/1038; B01J 35/1042; B01J 35/1061; B01J 37/0018; B01J 37/084; C01B 3/042; B01J 19/12; G01N 33/5005; G01N 21/6428; G01N 21/6402; B01J 2219/0892; B01J 2219/12; G01N 2021/6439; B01J 23/42

(57) ABSTRACT

Disclosed are compositions, processes, and methods directed to mesoporous carbon nitride materials having high nitrogen content. The mesoporous carbon nitride material has a three dimensional C₃N₅ 3-amino-1,2,4-triazole based mesoporous carbon nitride matrix having an atomic nitrogen to carbon ratio of 1.4 to 1.7, and a band gap of 1.8 to 3 eV.

### CROSS REFERENCE TO RELATED APPLICATIONS

This application claims the benefit of priority of U.S. Provisional Patent Application No. 62/375,022 filed Aug. 15, 2016, which is hereby incorporated by reference in its entirety.

### BACKGROUND

### 1. Field of the Invention

The invention generally concerns a three-dimensional nitrogen rich mesoporous material having a general formula of C₃N₆. In particular, the nitrogen rich mesoporous material includes a three dimensional (3D) C₃N₆ 3-amino-1,2,4-triazole based mesoporous carbon nitride matrix having an atomic nitrogen to carbon ratio of 1.4 to 1.7, and a band gap of 1.8 to 3 eV.

### 2. Description of Related Art

Scientific interest in carbon nitride (CN) materials has increased this last decade because of their unique semi-conductor behavior, basic sites, electronic properties, and other unique characteristics.

Mesoporous materials with three-dimensional (3D) porous structure have textural characteristics, such as specific surface areas, large pore volumes, and a unique 3D mesoporous channel network, which can provide a highly opened porous host with easy and direct access for guest species. 3D structures can facilitate easy inclusion or diffusion of reactant molecules throughout the pore channels without pore blockage (See, for example, Sakamoto et al., Nature, 2000, 408:449; Zhao et al., J. Am. Chem. Soc., 1998, 120:6024; Kim et al., J. Am. Chem. Soc., 2005, 127:7601; Alam et al., Chem. Asian J., 2011, 6:834).

Preparation of 3D graphitic mesoporous carbon nitrides have been prepared for use in sensor applications. By way of example, Vinu et al. (J. Mat. Chem A, Vol 1, (8), pp. 2913-2920) describes a hard template preparation of highly ordered and 3D graphitic mesoporous carbon nitride of 3-amino-1,2,4-triazine (MCN-ATN-x) for sensing acidic and basic organic vapors. The reported black carbon nitride material was prepared by an oxidation assisted route and has pore diameters of 5.5-6.0 nm. Korean Patent No. 101276612 to Hong et al. describes the synthesis of carbon nitride having 3D cubic form nanostructure for detection of copper ions. Anonietti et al. (Chem. Mater., Vol 23, (3), pp. 772-778) describes using carbon nitrides prepared from 1,2,4-triazoles catalysts for water splitting reactions. The carbon nitride material was prepared by ionothermal condensation of 3-amino-1,2,4-triazole-5-thiol using small quantities of MoCl₅ and a reactive cobalt precursor.

Many of the aforementioned catalysts suffer in that they are costly to manufacture and have limited chemical reactivity, light scattering, surface area, light absorption spectrum, recombination suppression properties, and/or have unordered structures. These deficiencies make the catalysts inefficient for sensing and/or solar energy conversion, and in particular, water splitting applications.

### SUMMARY

A discovery has been made that addresses the problems associated with catalysts for photocatalytic water splitting and sensing applications. The discovery is premised on the preparation of a nitrogen rich mesoporous material that includes a three dimensional (3D) C₃N₅ 3-amino-1,2,4-triazole (3-AT) based mesoporous carbon nitride matrix having a range of unique and beneficial properties. These properties include an atomic carbon to nitrogen (C:N) ratio of 0.5 to 0.7 (atomic N:C ratio of 1.4 to 2), preferably an atomic N:C ratio of 1.4 to 1.7, a band gap of 1.8 to 3 eV, a surface area of 250 to 325 m²/g, a pore volume of 0.2 to 0.6 cm³ g⁻¹, a pore size of 2 to 5 nm, or any combination thereof. Further characterization of the mesoporous material shows a highly basic, well ordered, 3)-cubic Ia3d symmetric mesoporous carbon nitride with graphitic pore walls and very high nitrogen content that exhibits unique semiconducting properties. Without wishing to be bound by theory, the combination of these properties along with facile preparation from readily available and nontoxic precursors makes the current mesoporous material suitable for applications in absorption of bulky molecules, catalysis, light emitting devices, photocatalytic water splitting, as a storage material, sensing device, solar cells, etc. Notably, the mesoporous material of the current invention have good textural properties and a narrow band gap without the addition of external semiconductor dopants (e.g., S, Ti, etc.) can be used to produce hydrogen (H₂) under visible light or to sense acidic acid and formic acid.

In a particular embodiment of the current invention, there is described a nitrogen rich mesoporous material. The nitrogen rich mesoporous material can include a three dimensional C₃N₅ 3-amino-1,2,4-triazole based mesoporous carbon nitride matrix having a carbon to nitrogen (C:N) ratio of 0.5 to 0.7 and a band gap of 1.8 to 3 eV. In one aspect, the mesoporous material includes a band gap of about 2.2 eV. Notably, the mesoporous material has a yellow color, which is in contrast to other known black 3-amino-1,2,4-triazole based mesoporous carbon nitride materials. Without wishing to be bound by theory, it is believed that the difference in the color of the mesoporous materials (e.g., yellow versus conventional black material) is due to the change in the arrangement of atoms in the CN walls of the 3D mesoporous carbon nitride. It is believed that the walls are composed of polytriazole framework instead of triazine framework, which are usually present in the C₃N₄ materials. It is also believed that this framework is responsible for the low band gap (2.2 eV) of the materials. In certain aspects, the material has a surface area of 250 to 325 m²/g, a pore volume of 0.2 to 0.6 cm³g⁻¹, a pore size of 2 to 5 nm, or any combination thereof. In some instances, the mesoporous material can also include a co-catalyst. The co-catalyst can include titanium, nickel, palladium, platinum, rhodium, ruthenium, tungsten, molybdenum, gold, silver, or copper, or combinations thereof, or alloys thereof. Specifically, the co-catalyst is platinum metal. A particular feature of the current invention is that the material is a photocatalytic active material.

According to another particular embodiment of the current invention, a photocatalytic process for producing hydrogen gas (H₂) from water is described. The process can include (a) contacting the mesoporous carbon nitride material described throughout the specification with water to form a reactant mixture; and (b) exposing the reactant mixture to light to form hydrogen gas from the water. In one aspect, the light source can be sunlight or a visible light source, or a combination thereof. Also disclosed is a C₁₋₂ hydrocarbon acid sensor that includes the mesoporous material of the current embodiments. The C₁₋₂ hydrocarbon acid of the sensor can include formic acid, acetic acid, or both.

In other embodiments, a method of producing the nitrogen rich mesoporous material of the current invention is described. The method can include: (a) obtaining an template reactant mixture that includes a calcined mesoporous KIT-6 template having a selected porosity and a protonated 3-amino-1,2,4-triazole; (b) heating the template reactant mixture to form a CN/KIT-6 composite; (c) heat treating the CN/KIT-6 composite to a temperature of 450° C. to 550° C. to form a cubic mesoporous carbon nitride material/KIT-6 complex; and (d) removing the KIT-6 template from the cubic mesoporous carbon nitride material/KIT-6 complex. The heating of step (b) can include heating to a first temperature of 90° C. to 110° C., preferably about 100° C. for 4 to 8 hours, preferably, 6 hours; and increasing the temperature to 150 to 170° C., preferably about 160° C. for 4 to 8 hours, preferably 6 hours. In one aspect, the heat treating temperature is about 500° C. In another aspect, the CN/KIT-6 composite can be heated under an inert gas atmosphere (e.g., argon). In some instances, the template reactant mixture can include adding calcined KIT-6X to an aqueous solution of 3-amino-1,2,4-triazole and hydrochloric acid. The method can further include producing the KIT-6 template by (a) obtaining a polymerization solution that includes an amphiphilic triblock copolymer and tetraethyl orthosilicate (TEOS); (b) reacting the polymerization mixture at a predetermined reaction temperature to form a KIT-6 template, where the predetermined temperature determines the pore size of the KIT-6 template; (c) drying the KIT-6 template at 90° C. to 110° C., preferably 100° C.; and (d) calcining the dried KIT-6 template in air at 500 to 600° C., preferably 540° C. to form the calcined KIT-6 template. The polymerization mixture can then be incubated (held) at a synthesis temperature of about 100 to 200° C., preferably 150° C.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The following includes definitions of various terms and phrases used throughout this specification.

The phrase “nitrogen rich” refers to carbon nitrides having more nitrogen atoms than graphitic carbon nitrides having the general formula of C₃N₄.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The 3-D mesoporous CN materials of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the 3-D mesoporous CN materials of the present invention are their capabilities to catalyze photocatalytic water-splitting reactions and to sense organic acids.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein. The drawings may not be to scale.

FIG. 1 is a schematic representation for the preparation of 3-amino-1,2,4-triazole based 3D cubic mesoporous carbon nitride (MCN-TZL) of the present invention using KIT-6.

FIG. 2 shows the low angle powder X-ray diffraction (XRD) patterns of KIT-6 and MCN-TZL of the present invention.

FIG. 3 shows the wide angle powder XRD pattern of MCN-TCL of the present invention.

FIG. 4 shows nitrogen adsorption/desorption isotherms of KIT-6 and MCN-TZL of the present invention.

FIG. 5 shows adsorption pore size distributions of KIT-6 and MCN-TZL of the present invention determined using Brunauer-Emmett-Teller (BET) methodology.

FIG. 6A shows transmission electron microscopy (TEM) images of MCN-TZL of the present invention.

FIG. 6B shows scanning electron microscopy (SEM) images of MCN-TZL of the present invention.

FIG. 7 shows an electron energy loss (EEL) spectrum of MCN-TZL of the present invention.

FIG. 8 shows the Energy Dispersive X-ray (EDX) analysis and corresponding elemental mapping of C and N elements in MCN-TZL of the present invention.

FIG. 9 shows X-ray photoelectron survey spectrum (XPS) of MCN-TZL material of the present invention

FIG. 10 show C1s XPS of MCN-TZL material of the present invention.

FIG. 11 shows N1s XPS of MCN-TZL material of the present invention.

FIG. 12 shows Fourier transformed infrared (FTIR) spectra of 3-AT and MCN-TZL of the present invention.

FIG. 13 shows the UV-Vis absorption spectrum of MCN-TZL of the present invention.

FIG. 14 shows the photoluminescence spectrum of MCN-TZL of the present invention.

FIG. 15 shows the time course of hydrogen evolution obtained over MCN-TZL material of the present invention under visible light irradiation

FIG. 16 shows the response of a MCN-TZL of the present invention coated QCM sensor to the exposure of different organic vapors.

FIG. 17 shows bar graphs of frequency shifts for the different vapors detected by the QCM senor in FIG. 16.

FIG. 18 shows the Temperature Programmed Desorption (TPD) plot of carbon dioxide desorbed from the MCN-TZL sample of the present invention.

FIG. 19 shows measurements done on the bare QCM electrode.

### DETAILED DESCRIPTION

A discovery has been made that provides a mesoporous carbon nitride (CN) material having the appropriate characteristics for photocatalytic water-splitting and sensing applications. The discovery is premised on a preparation method that produces a highly nitrogen rich CN material having suitable pore diameters and band gap to achieve high photon absorption relative to photon energy. In certain aspects, the tuning of the mesoporous CN material is accomplished by controlling the pore size and other dimensions of the mesoporous CN material.

These and other non-limiting aspects of the present invention are discussed in further detail in the following sections with reference to the Figures.

### A. Mesoporous Carbon Nitride Materials

Certain embodiments are directed to a nitrogen rich mesoporous material based on 3-amino-1,2,4-triazole (3-AT). Such a material can have a 3-D body-centered cubic structure and have a general formula of C₃N₅(designated as MCN-TZL throughout the specification). The MCN-TZL can have a pore size or pore diameter of 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, nm, 21 nm, 22 nm, 23 nm, 24 nm, 25 nm, 26 nm, 27 nm, 28 nm, 29 nm, or 30 nm. Specifically, the pore size can range from 2 nm to 10 nm, preferably 2 nm to 5 nm. In certain aspects the mesoporous material can have an atomic nitrogen to carbon (N:C) ratio greater than, equal to or between any two of 1.4, 1.5, 1.6, 1.7, 1.8, 1.9 and 2.0. Specifically the atomic N:C ratio can range from 1.4 to 1.95. In a specific embodiment, the atomic N:C ratio is 1.5 to 1.7. The pore volume of the mesoporous material can range from 0.1 to 1 cm³g⁻¹ or any value or range there between (e.g., 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.40, 0.41, 0.42, 0.43, 0.5, 0.51, 0.52, 0.53, 0.54, 0.55, 0.56, 0.57, 0.58, 0.59, 0.60, 0.61, 0.62, 0.63, 0.64, 0.65, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, or 0.99, cm³g⁻¹). Preferably, the pore volume is 0.2 cm³g⁻¹ to 0.6 cm³g⁻¹. The surface area of the MCN-TZL can be from 200 to 400 m² g or great than, equal to, or between any two of 200, 210, 220, 230, 240, 250, 260, 270, 280, 290, 300, 320, 330, 340, 350, 360, 370, and 400 m²g⁻¹. Preferably, the surface area is from 225 m²g⁻¹ to 350 m²g⁻¹ or from 250 m² g⁻¹ to 325 m²g⁻¹. In certain aspects, the MCN-TZL material can be tuned to a low band gap of 1.8 to 3 eV, or 1.8 to 2.5 eV, 1.9 to 2.4 eV, or 1.8, 1.9, 2.0, 2.1, 2.2, 2.4, 2.5, 2.6, 2.7, 2.8, 2.9, or 3.0 eV. Preferably, the band gap is about 2.2 eV. Without being limited by theory, the MCN-TZL material of the present invention has highly basic characteristics that provide its unique and beneficial properties.

The photocatalytic reaction of MCN-TZL materials of the present invention can be assisted by the addition of metals, which can serve as a co-catalyst in a water splitting reaction. In certain aspects, the co-catalyst is or includes a metal such as titanium, nickel, palladium, platinum, rhodium, ruthenium, tungsten, molybdenum, gold, silver, copper, or combinations thereof. Preferably, the co-catalyst is platinum metal. The MCN-TZL material can include 0.01, 0.02, 0.03, 0.04, 0.05, 0.06, 0.07, 0.08, 0.09, 0.10, 0.11, 0.12, 0.13, 0.14, 0.15, or 2.0 wt. % of the co-catalyst. In certain aspects, the co-catalyst is incorporated on the surface of or embedded in the MCN-TZL material. The photon energy necessary to split water is greater than 1.23 eV, thus tuning the band gap of the mesoporous CN material can allow for more water splitting than recombination. Without wishing to be bound by theory, it is believed that tuning the CN band gap reduces the likelihood that an excited electron will spontaneously revert to its non-excited state (i.e., the electron-hole recombination rate can be reduced or suppressed). When the MCN-TZL material is irradiating with light (i.e., sunlight or visible light) an electron can move from a given valence band (VB) to a given conduction (CB) (e.g., excitation through absorption of light), the electron will be restrained from spontaneously moving back to the VB, as the spontaneous emission of a photon that is typically associated with such a move from the CB to the VB would be at a frequency that is restricted due to the material's photonic band gap. The electron can remain in the CB for a longer period of time, which can result in use of said electron to split water rather than moving back to its VB (i.e., the electron-hole pair remains in existence for a longer period of time). This, coupled with the electrically conductive material (co-catalyst) deposited on the photoactive material, provides for a more efficient use of the excited electrons in water-splitting applications. The co-catalyst can be an electron sink and/or promote H₂ production from water instead of electron-hole (e⁻-h⁺) recombination events during the photocatalytic water-splitting reaction. In some aspects where the co-catalyst is platinum metal, the platinum can function as an efficient H₂ evolution promoter due to small over-potential and depression of radiative recombination of photo-induced charge carriers.

### B. Method of Making

The MCN-TZL material can be formed by nanocasting using a template. Nanocasting is a technique to form periodic mesoporous framework using a hard template to produce a negative replica of the hard template structure. A molecular precursor can be infiltrated into the pores of the hard template and subsequently polymerized within the pores of the hard template at elevated temperatures. Then the hard template can be removed by a suitable method. This nanocasting route is advantageous because no cooperative assembly processes between the template and the precursors are required. A hard template can be a mesoporous silica. In one aspect, the mesoporous silica can be KIT-6, MCM-41, SBA-15, TUD-1, HMM-33, etc., or derivatives thereof prepared in similar manners from tetraethyl orthorsilicate (TEOS) or (3-mercaptopropyl) trimethoxysilane (MPTMS). In certain aspect, the mesoporous silica is a 3D-cubic Ia3d symmetric silica, such as KIT-6 which contains interpenetrating cylindrical pore systems. Highly ordered mesoporous silicas can be obtained under various conditions using inexpensive materials.

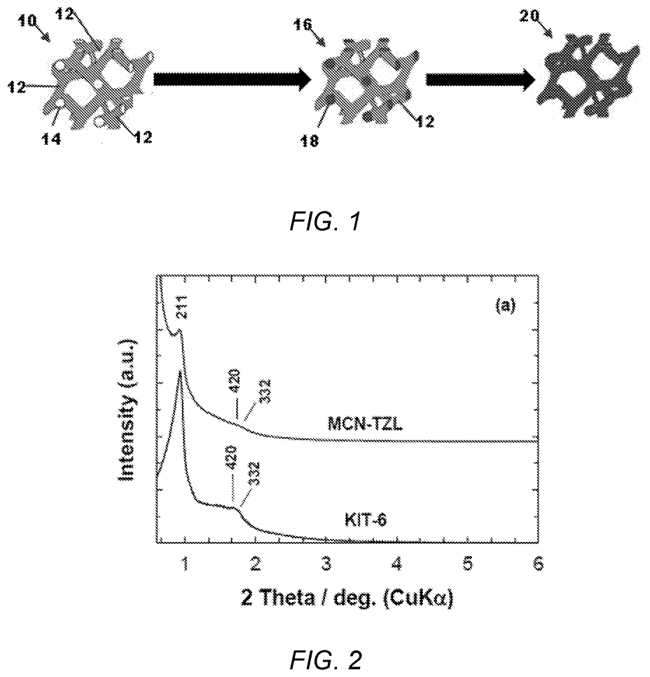

FIG. 1 is a schematic representation of one embodiment of a method for producing a MCN-TZL (C₃N₆) material by using a hard templating approach, also called a replica approach, as described herein. Template 10 (e.g., calcined KIT-6) can include canal 12 and pores 14. Canal 12 is representative of the pore volume of template 10. Pores 14 can be filled corresponding carbon nitride precursor material 16 to form a template/carbon nitride precursor material. By way of example, an aqueous solution of 3-amino-1,2,4-triazole can be added to a KIT-6. The template/carbon nitride precursor material can undergo a thermal treatment to polymerize the precursor inside the pore of the material to form template/CN composite 16 having canal 12 and polymerized CN material 18. Template/CN composite 16 can be subjected to conditions sufficient to dissolve the template 10 (e.g., KIT-6), and form mesoporous carbon nitride material 20 of the present invention. By way of example, template 10 can be dissolved using an HF treatment, a very high alkaline solution, or any other dissolution agent capable of removing the template and not dissolving the CN framework. The kind of template and the CN precursor used influence the characteristics of the final material. By way of example, various KIT-6 with various pore diameters can be used as templates. In certain aspects, the pore size of the KIT-6 template can be tuned and 3-amino-1,2,4-triazole can be used to produce a high nitrogen content.

In one non-limiting embodiment, step one of a method to prepare a nitrogen rich mesoporous material can include obtaining an template reactant mixture including a calcined mesoporous KIT-6 template having a selected porosity and a protonated 3-amino-1,2,4-triazole (3-AT). In some instances, obtaining the template reactant mixture includes adding calcined KIT-6 to an aqueous solution of 3-amino-1,2,4-triazole and hydrochloric acid. In other instances, the template reactant mixture can be a gel. In step 2 of the method, the template reactant mixture can be heated to form a CN/KIT-6 composite. The heating of the template reactant mixture to form a composite can include heating to a first temperature of 90 to 110° C., preferably about 100° C. for 4 to 8 hours, preferably, 6 hours and increasing the temperature to 150 to 170° C., preferably about 160° C. for 4 to 8 hours, preferably 6 hours. Step 3 of the method includes polymerization of the CN/KIT-6 composite. The CN/KIT-6 composite can be heated under a flow of inert atmosphere (i.e. argon) to a temperature of 450 to 550° C., preferably about 500° C., for a period of time to form a cubic mesoporous carbon nitride material/KIT-6 complex. In some aspects, the CN/KIT-6 composite can be heated under inert atmosphere gas flow to temperature at a rate of about 1, 2, 3, 4, 5, or 6° C. per minute. The inert atmosphere gas flow can be at about 50, 60, or 70 to 100, 120, or 150 ml per minute, including all values and ranges there between. In step 4 of the method, the KIT-6 can be removed by dissolving the KIT-6 template from the cubic mesoporous carbon nitride material/KIT-6 complex to form the MCN-TZL material of the present invention. In some aspects, hydrofluoric acid or other suitable solvent or treatment can be used that dissolves the KIT-6 without dissolving the CN framework. The method can further include collecting the cubic mesoporous carbon nitride material by filtration. In a further aspect, the filtered material can be ground to a powder and/or purified and/or stored and/or used directly in subsequent applications (i.e., photocatalytic or sensing applications).

A non-limiting example of producing a MCN-TZL material includes mixing 3-amino-1,2,4-triazole (3-AT) in aqueous HCl with stirring at 35° C. Once dissolution is achieved, the mixture can be placed in a drying oven for 6 hours at 100° C. and then 160° C. for another 6 hours. The resulting silica template and practically condensed 3-AT can then be heat treated at 500° C. under inert atmosphere. The composite obtained after carbonization was treated with HF at room temperature to dissolve the silica template. The template free carbon nitride obtained can be filtered, washed several times with ethanol, and dried at 100° C. to afford a yellow MCN-TZL solid material. The MCN-TZL of the present invention is yellow in color and in powder form.

In some aspects, the MCN-TZL material can include a metal or metal alloy as a co-catalyst. The metal or metal alloys can be obtained from a variety of commercial sources in a variety of forms (e.g., particles, rods, films, etc.) and sizes (e.g., nano scale or micro scale). By way of example, each of Sigma-Aldrich® Co. LLC and Alfa Aesar GmbH & Co KG offer such products. Alternatively, the metal containing MCN-TZL can be prepared using co-precipitation or deposition-precipitation methods. The metal can be deposited on the MCN-TZL material prior to or during a photochemical reactions. By way of example, a metal precursor (e.g., a metal nitrate or metal halide) can be added to an aqueous solution containing the MCN-TZL material and a sacrificial agent. The metal salt can absorb on the surface of the MCN-TZL material. Upon irradiation, the metal ions can be converted to the active metal species (e.g., zero valance).

A KIT-6 template can be produced by in the following manner and the methods exemplified in the Example section. An amphiphilic triblock copolymer can be dispersed in an aqueous hydrogen chloride solution with 1-butanol and tetraethyl orthosilicate (TEOS) to form a polymerization mixture. The polymerization mixture can be held (incubated) at a predetermined synthesis temperature to form a KIT-6 template. The KIT-6 template material can be calcined at 500 to 600° C. or any value or range there between (e.g., 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 543, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, or 599° C., preferably 540° C.). The polymerization mixture can be incubated at a synthesis temperature of about 100 to 200° C., or any value or range there between (e.g., 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 143, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199° C.). The temperature can be selected can be used to tune the pore size of the KIT-6 template. In the general formula KIT-6-X, X denotes the incubation temperature. For the general formula KIT-6-X, X represents the incubation temperature. For example, in certain aspects the polymerization mixture can be incubated at a synthesis temperature of about 100, 130, or 150° C. to yield corresponding KIT-6 templates, denoted KIT-6-100, KIT-6-130, and KIT-6-150 respectively.

A non-limiting example of producing a KIT-6 template includes mixing Pluronic P-123 in aqueous HCl with stirring at 35° C. until dissolution. n-Butanol can then be added with continued stirring and after 1 hour TEOS can be added and the resulting mixture can be vigorously stirred at 35° C. for 24 hours. The mixture can then be aged (incubated) at 150° C. for 24 h under static conditions and the resulting colorless solid can then be filtered at 50° C. or less without washing. The resulting solid can be dried in oven at 100° C. for 24 h, and then calcined in air at 540° C.

### C. Use of the Mesoporous Carbon Nitride Materials

The three dimensional C₃N₆ 3-amino-1,2,4-triazole based mesoporous carbon nitride matrix material with or without a co-catalyst can be used in applications for absorption of bulky molecules, catalysis, light emitting devices, photocatalytic water splitting, as a storage material, sensing device, solar cells, etc. Specifically, the mesoporous material of the current invention can be used to produce hydrogen (H₂) under visible light or to sense acidic acid and formic acid.

In one non-limiting aspect, MCN-TZL has good photoluminescence and can be used as a photocatalyst in a photocatalytic process for producing hydrogen gas (H₂) from water in a water-splitting reaction. The process can include (a) contacting the mesoporous material with water to form a reactant mixture; and (b) exposing the reactant mixture to light (e.g., sunlight, visible light, or a combination thereof) to form hydrogen gas from the water. The produced hydrogen gas can be purified and/or stored and/or used direction in subsequent reactions (e.g., hydrogenation reactions). Without wishing to be bound by theory, it is believed that the three dimensional C₃N₆ 3-amino-1,2,4-triazole based mesoporous carbon nitride matrix material fulfills the three main requirements for a water splitting photocatalyst of: (i) an oxidative active site for the oxygen evolution, (ii) a reductive site for the hydrogen generation, and (iii) a good semi-conductor for the photon absorption.

In some embodiments, a sacrificial agent can be added to the reactant mixture. The presence of the sacrificial agent can increase the efficiency of the photosystem by further reducing the likelihood of hole/electron recombination via oxidation of the sacrificial agent by the hole rather than recombination with the excited electron and/or assist in photodeposition of the co-catalyst on the MCN-TZL surface. Non-limiting examples of sacrificial agents that can be used in the methods of the present invention include ethanolamines, alcohols, diols, polyols, dioic acids, or any combination thereof. A non-limiting example of a particular sacrificial agent includes triethanolamine.

In another non-limited aspect, MCN-TZL of the present invention enhanced sensing properties. MCN-TZL can be included in a C₁₋₂ hydrocarbon acid sensor, such as for detecting formic acid, acetic acid, or both. In another aspect, MCN-TZL can be used as a biosensor. The MCN-TZL can be filled with a fluorescent dye that would normally be unable to pass through cell walls. The MCN-TZL material can then be capped off with a molecule that is compatible with the target cells. When the capped MCN-TZL material is added to a cell culture, they can carry the dye across the cell membrane. In some instances, the MCN-TZL material can be optically transparent, so the dye can be seen through the silica walls. Encapsulating the dye within the MCN-TZL material can inhibit self-quenching of the dye.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Materials. Tetraethyl orthosilicate (TEOS), 3-amino-1,2,4-triazole (3-AT), n-butanol, and triblock copolymer poly(ethylene glycol)-block-poly(propylene glycol)-block-poly(ethylene glycol) (Pluronic P-123, molecular weight 5800 g mol⁻¹, EO₂₀PO₇₀EO₂₀) were obtained from Sigma-Aldrich® (U.S.A). Ethanol and hydrofluoric acid (HF) were purchased from Wako Pure Chemical Industries (U.S.A.). All the chemicals were used without further purification. Doubly deionized water was used throughout the synthesis process.

### Example 1

### Synthesis of Three-Dimensional Cubic Mesoporous Silica Template, KIT-6

Pluronic P-123 (4.0 g) was dissolved in a solution containing distilled water (144 g) and HCl (36 wt. %, 7.9 g) with stirring at 35° C. After complete dissolution, n-butanol (4.0 g) was added immediately. After stirring 1 h, TEOS (8.6 g) was added to the homogeneous clear solution with constant agitation. The mixture was kept under vigorous and constant agitation at 35° C. for 24 hours. Subsequently, the reaction mixture was aged at 150° C. for 24 h under static conditions. The molar gel composition of the synthesis mixture was 0.041TEOS:0.0007P123:0.054C₄H₉OH:0.076HCl:8.28 H₂O. The white solid product was filtered hot without washing, dried in oven at 100° C. for 24 h, and then calcined in air at 540° C.

### Example 2

### Synthesis of Three-Dimensional Mesoporous Carbon Nitride MCN-TZL from 3-AT

3-amino-1,2,4-triazole (3.0 g, 3-AT) and KIT-6 (1.0 g) was mixed in acidic DI water (0.16 g HCl in 4-5 g DI water). The mixture agitated for few minutes at a few degrees above room temperature. Upon complete dissolution, the mixture was placed in a drying oven for 6 hours at 100° C. and then 160° C. for another 6 hours. The composite of silica template and partially condensed 3-AT was heat treated at 500° C. in argon atmosphere. The composite obtained after carbonization was treated with HF at room temperature to dissolve the silica template. The template free carbon nitride (MCN-TZL) obtained was filtered, washed several times with ethanol, and dried at 100° C.

### Example 3

### Characterization of MCN-TZL and KIT-6

### 1. X-Ray Diffraction Analysis

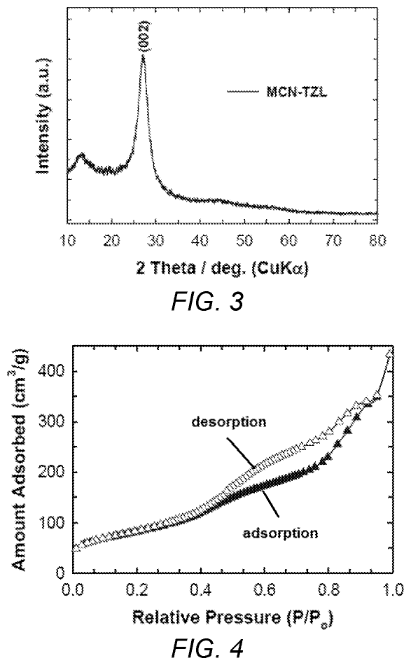

XRD: Powder XRD patterns were recorded on a Rigaku Ultima+(JAPAN) diffractometer using CuKα (λ=1.5408 Å) radiation. Low angle powder x-ray diffractograms were recorded in the 2θ range of 0.6-6° with a 2θ step size of 0.0017 and a step time of 1 sec. In case of wide-angle X-ray diffraction, the patterns were obtained in the 20 range of 10-80° with a step size of 0.0083 and a step time of 1 sec. FIG. 2 shows the low angle powder XRD patterns of MCN-TZL of the present invention and the 3D mesoporous silica template, KIT-6. FIG. 3 is a wide-angle powder XRD pattern of MCN-TZL of the present invention.

The XRD pattern of the KIT-6 silica template exhibited a sharp well resolved (211) reflection and several higher order reflections, (420), (332) at 2θ angles below 4°, indicating long range structural ordering with the symmetry of body centered cubic Ia3d space group. The unit cell constant, calculated from the (211) reflection using the equation d₂₁₁ \sqrt{6}, was found to be 23.1 nm.

The XRD pattern of the MCN-TZL showed a well-resolved peak with d-spacing of 9.03 nm along with several weak higher order reflections. The highly intense peak was indexed to (211) reflection of the cubic type Ia3d structure, almost similar to the parent mesoporous silica template, KIT-6. The unit cell parameter from the (211) reflection was measured to be 23.0 nm, which was slightly lower than that of the parent template. This was attributed to a little structural shrinkage of the mesoporous structure of [3AT-KIT-6] nanocomposite materials either during the pyrolysis at 500° C. or during silica removal process by strong HF. Noteworthy, the intensity of main peak of the XRD pattern of MCN-TZL was smaller than that of the KIT-6 template, suggesting the defects in the pore walls. From the XRD it was determined that the MCN-TZL possesses 3D cubic structure with an enantiomeric system of independently interpenetrating continuous network of mesoporous channels. The crystallinity and graphitic character of the mesoporous wall structure, the MCN-TZL material was characterized by wide angle XRD analysis (FIG. 3). FIG. 3 shows a more pronounced diffraction peak at 2θ=27.2°, corresponding to an interlayer d-spacing of 3.27 Å, which was indexed to the (002) reflection of the pure graphitic lattice, thereby confirming the turbostratic ordering in the wall structure. The large intensity of the (002) reflection was attributed to the graphitic ordering (partial crystallinity) in the pore walls. In addition, weak peak at 13.4° (d spacing=6.58 Å) was attributed to in-plane structural ordering.

### 2. Textural Parameters and Elemental Analysis

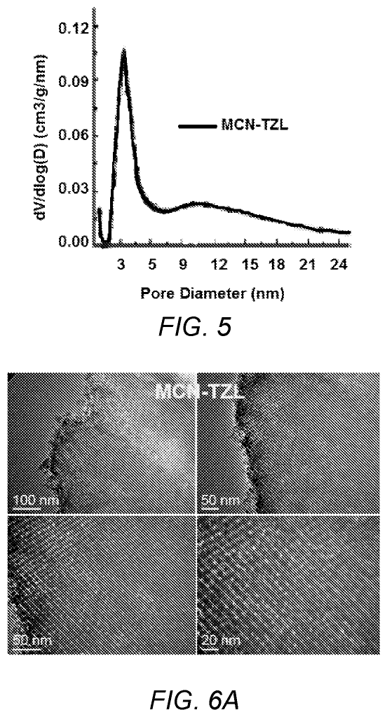

Textural parameters and chemical analysis. Textural parameters and mesoscale ordering of the MCN-TZL material was confirmed by nitrogen adsorption/desorption measurements using a Quantachrome Instruments (U.S.A.) sorption analyzer at −196° C. All samples were out-gassed for 12 hrs at high temperatures under vacuum (p<1×10−5 h.Pa) in the degas port of the adsorption analyzer. The specific surface area was calculated using the Brunauer-Emmett-Teller (BET) method. The pore size distributions were obtained from either adsorption or desorption branches of the isotherms using Barrett-Joyner-Halenda (BJH) method. FIG. 4 shows the N₂ adsorption-desorption isotherms of the MCN-TZL. The isotherm was of type IV according to IUPAC classification and with characteristic capillary condensation or evaporation step, which indicated the presence of a well-ordered mesoporous structure. MCN-TZL showed narrow pore size distribution centered at 3.42 nm (FIG. 5). Specific surface area (S_{BET}), pore volume (P_{V}) and pore diameter (P_{D}) values along with lattice constant (a₀) of MCN-TZL and sacrificial template, KIT-6 are listed in Table 1.

Chemical analysis was carried out by using a Yanaco MT-5 CHN elemental analyzer (Yanaco Bunseki Kogyo Co., JAPAN). The carbon to nitrogen ratio of the material was found to be about 0.64 with 2.05 wt. % of hydrogen, which was in close agreement with the values obtained from the EDX and XPS analysis below. The high nitrogen content detected in the sample as compared with the ideal C₃N₄(ca. 0.73) structure was attributed to the increased number of amine groups attached to the ring structure in MCN-TZL.

TABLE 1

| | | | | | Elemental composition | | | |
|---|---|---|---|---|---|---|---|---|
| | | | Pore | Pore | by CHN analysis | | | |
| | a₀ | A_{BET} | Volume, | Diameter, | C | N | H | |
| Sample Name | (nm) | (m²/g) | Pv (cm³/g) | P_{D} (mm) | (wt. %) | (wt. %) | (wt. %) | C/N |
| KIT-6-150 | 23.1 | 559.1 | 1.46 | 9.92 | — | — | — | — |
| MCN-TZL | 23.0 | 296.7 | 0.53 | 3.42 | 31.22 | 48.51 | 2.05 | 0.64 |

*Cell parameters were calculated from low-angle XRD patterns (FIG. 2) using a₀ = √6*d₂₁₁.
Total pore volumes were estimated from the adsorbed amount at a relative pressure of p/p0 = 0.99. Pore diameters derived from the adsorption branches of the isotherms by using the BJH method.

From the data it was determined that even after the strong HF treatment for removing silica, the MCN-TZL material retained its mesoporous structure with high specific surface area (296.7 m²/g) and large pore diameter (3.42 nm). The specific surface area of MCN-TZL was lower than that of KIT-6 mainly because of lack of microporosity.

### 3. HRTEM, FESEM, EDX and EELS

FESEM and EDX: Morphology of the samples was observed on a Hitachi S-4800 (U.S.A.) field emission scanning electron microscope (FE-SEM). The machine is equipped with energy dispersive X-ray (EMAX) elemental analyzer. Prior to observation, all the samples were sputtered with Pt for 20 sec by using ion coater. Samples were measured under the accelerating voltage of 5-10 kV, emission current around 10 mA condensed lens of Megapixel. For SEM, objective aperture 2 was used with working distance around 8 mm while during elemental analysis (EDX), aperture number 1 with working distance around 15 mm was used. EDX along with elemental mapping were recorded on the same machine using accelerating voltage of 15 kV.

HRTEM and EELS: HRTEM images were obtained using a JEOL-3100FEF (JOEL, U.S.A.) high-resolution transmission electron microscope, equipped with a Gatan-766 electron energy-loss spectrometer (EELS). The preparation of the samples for HRTEM analysis involved sonication in ethanol for 5 min and deposition on a copper grid. The accelerating voltage of the electron beam was 200 kV.

FIG. 6A shows the TEM images of MCN-TZL at various magnifications. Well-ordered arrays of pore channels were observed. From the images, it was determined that pore size and connectivity of the material exactly reflected the geometric properties of the original template, KIT-6. Bright contrast strips on the images represent the pore-wall images, whereas dark contrast cores display empty channels. The contrast pattern can be converted by changing the lens defocus. The image taken at low magnification revealed the highly ordered mesoporous structure and the pores are highly organized whereas the high resolution image displays the interconnection between the pores and the three dimensional orientation of the mesopores. The porous structure, when viewed in the direction perpendicular to their axis, showed a linear array of mesopores that are arranged in patches composed of regular rows more than 1 m long. FIG. 6B shows the SEM images of the MCN-TZL. The sample exhibits sheet like morphology, composed of ultra-fine CN nanoparticles.

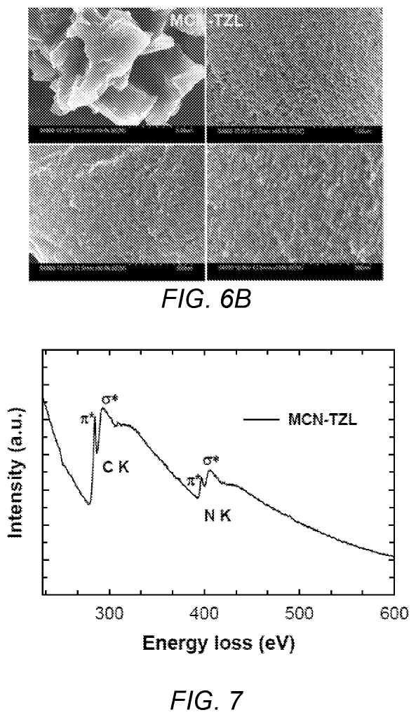

FIG. 7 shows the electron energy loss (EEL) spectrum recorded for MCN-TZL sample. The sample exhibited identical, well resolved carbon K-ionization and nitrogen K-ionization edges located at 284.6 eV and 401 eV, respectively, which indicated a similar electronic environment of C and N in the material. The peak at 284.6 eV was due to 1s-π* electron transitions which indicate the presence of sp² hybridized carbon bonded to nitrogen while the signal at 401 eV was assigned to the sp² hybridized nitrogen atoms which were present with carbon atoms in the wall structure. From the spectrum, it was clear that the MCN-TZL framework was entirely composed of C and N atoms and no impurities were detected. However, the presence of oxygen was below the detection limit, which supported the fact that trace amounts of oxygen found in EDX analysis were not intrinsic components of the product. The origin of oxygen can be atmospheric moisture or adsorbed CO₂, or H₂O that were removed under the electron beam irradiation and high vacuum conditions of the electron microscope.

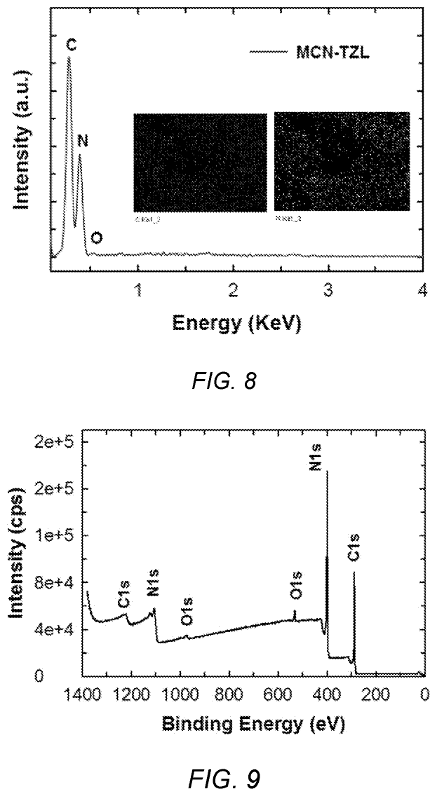

FIG. 8 show the Energy Dispersive X-Ray (EDX) spectrum of MCN-TZL. Peaks for the elements C, N, O were identified in the EDX spectrum. The absence of the signals at Si or F positions indicated effective silica removal by strong acid, HF. The weight and atomic percent of carbon, nitrogen and oxygen is listed in Table 2 from which it was determined that the MCN-TZL had an atomic C/N ratio of (ca. 0.59).

TABLE 2

| Element | Weight % | Atomic % |
|---|---|---|
| CK | 33.26 | 36.79 |
| NK | 65.81 | 62.43 |
| OK | 0.93 | 0.78 |

### 4. UV-Vis

UV-Vis spectra of the MCN-TZL sample was obtained using a LAMBDA 750 UV/VIS/NIR spectrophotometer (190 nm-3300 nm) from Perkin Elmer (U.S.A.). Instrument is equipped with a diffuse reflectance integrating sphere coated with BaSO4, which serve as a standard. Thickness of the quartz optical cell was 5 mm. The band gap of the materials were calculated using Tauc Plot method. FIG. 13 shows the UV-Vis absorption spectrum of MCN-TZL. Intrinsic absorption edge of MCN-TZL covered longer wavelengths of the visible region, which can be attributed to small band-gap energy. The band gap energy (E_{g}) of MCN-TZL was estimated to be 2.20 eV using Tauc Plot method. Such a band-gap was attributed to intrinsic semiconductor like absorption properties. Band gap value of MCN-TZL was much smaller than the reported band gap energies of mesoporous graphitic C₃N₄ or doped C₃N₄ compounds fabricated with different precursors (2.6 to 2.9 eV). Nevertheless, the band gap of MCN-TZL was sufficiently large to overcome the endothermic barrier for water splitting (1.23 eV, theoretically). Without wishing to be bound by theory, it is believed that the optical band gap can be attributed to the transitions between the weakly localized π-π* states that comes from the sp² hybridization atoms in the network. These states form the valence and conduction band edges and in turn control the width of the band gap. The π bonding at the C sp² site favored the clustering of the aromatic rings into graphitic sheets, as was evidenced from the wide angle XRD pattern of MCN-TZL.

### 5. XPS, FTIR, and UV-Vis Diffuse Reflectance Spectroscopy

XPS spectra of the MCN-TZL sample was obtained using a PHI Quantera SXM (ULVAC-PHI, JAPAN) instrument with a 20 kV, Al Kα probe beam (E=1486.6 eV). Prior to the analysis, the samples were evacuated at high vacuum (4×10⁻⁷ Pa), and then introduced into the analysis chamber. For narrow scans, analyzer pass energy of 55 eV with a step of 0.1 eV was applied. To account for the charging effect, all the spectra were referred to the C1s peak at 284.5 eV. Survey and multiregion spectra were recorded at C1s and N1s photoelectron peaks. Each spectral region of photoelectron interest was scanned several times to obtain a good signal-to-noise ratio. FTIR spectra of the 3-AT (precursor) and MCN-TZL (product) was obtained using a Perkin Elmer (U.S.A.) spectrum 100 series, bench top model equipped with the optical system that gives the data collection over the range of 7800 to 370 cm⁻¹. The spectra were recorded by averaging 200 scans with a resolution of 2 cm⁻¹, measuring in transmission mode using the KBr self-supported pellet technique. The spectrometer chamber was continuously purged with dry air to remove water vapor. The nature and coordination of the carbon and nitrogen atoms in the MCN-TZL sample analyzed using XPS and FTIR.

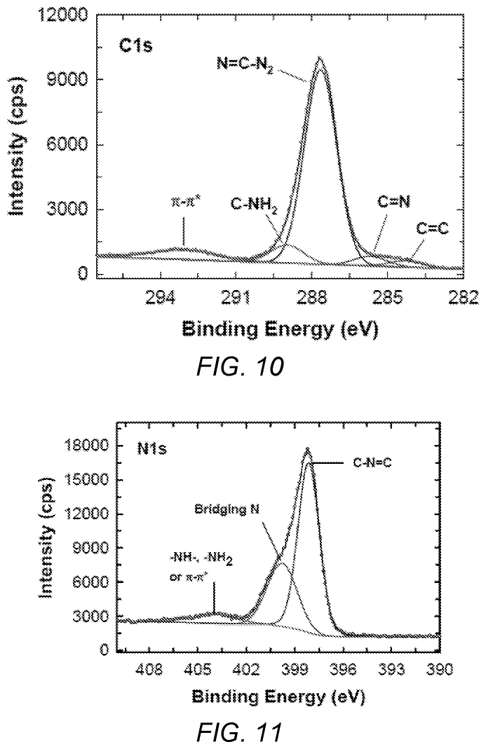

From the XPS survey spectrum of the MCN-TZL material (FIG. 9), it was determined that the material was composed of C and N with a small amount of oxygen. As explained above in the EELS discussion, the presence of oxygen was attributed to the atmospheric moisture or CO₂ adsorption on the surface. The C1s and N1s spectra are shown in FIG. 10 and FIG. 11 respectively. In order to know the details of the chemical bonding between the C and N atoms in the CN matrix, C1s and N1s peaks were deconvoluted into Gaussian-Lorenzian shapes. The C1s peak was deconvoluted into four peaks with binding energies of 289.1, 287.7, 285.7, and 284.3 eV. The N1s peak was deconvoluted into three peaks at binding energies of 398.2, 399.9 and 403.8 eV. The peak in the C1s spectrum centered at 287.7 eV was very sharp with large intensity. This peak was assigned to the carbon atoms bonded with three N neighbors (N₂—C═N—). The least intense peak, positioned at 284.3 eV, were assigned to the C—C bond due to carbon contamination or adventitious carbon, and whereas the peak at 285.7 eV was attributed to the sp² C atoms bonded to N inside the aromatic structure. The highest energy contribution at 289.1 eV was assigned to the sp²-hybridized carbon in the aromatic ring attached to the NH₂ group. These assignments are in good agreement with the values reported for nonporous carbon nitride samples. The N1s spectra of the samples in the FIG. 11 revealed the presence of two kinds of nitrogen atom in MCN-TZL whose components have peaks at about 398.2 and 399.9 eV. The lowest energy contribution, 398.2 eV, of the N1s spectrum was attributed to nitrogen bonded with two carbon atoms in a graphitic sp² network. While the peak positioned at 399.9 eV corresponds to the small fraction of bridging nitrogen atoms such as —N<(tertiary), —NH— (secondary), or —NH₂ (primary) In addition, the weak and broad signal detected around 293.1 eV in C1s and 403.8 eV in N1s spectrum was assigned to the n electron delocalization in CN heterocycles or other nitrogen functionalities like primary or secondary amines.

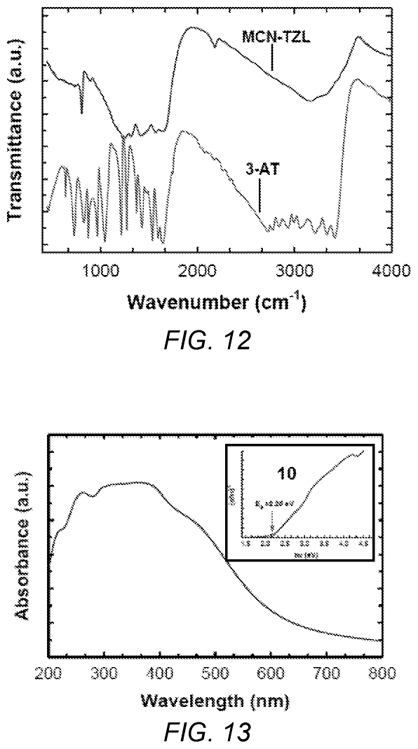

FIG. 12 shows FT-IR spectrum of 3-AT (precursor) and MCN-TZL (product). The spectrum for 3-AT shows bands at 3100-3400 cm⁻¹ were characteristic of —NH, NH₂ stretching and bands at 1647 cm⁻¹ corresponded to the bending mode of NH₂ vibrations (6-NH₂). A strong signal at 1425 cm⁻¹ indicated the ring breathing modes of the triazole nucleus and absorption in the range 1270-1300 cm⁻¹ was attributed to the secondary aromatic amines and C—N bonds. The strong bands in the 1000-700 cm⁻¹ region were attributed to the N—H out-of-plane bending vibrations and (ω-NH) vibrations. The spectrum of MCN-TZL shows broad bands at 3100-3400 cm⁻¹, which corresponded to the stretching modes of NH₂, or NH groups, which indicated that even after high temperature treatment, amines were retained in the final structure to serve as bridging centers in the final structure. The peaks in the 1100-1400 cm⁻¹ and 1500-1600 cm⁻¹ regions represented modes involving C—N and C═N stretching vibrations, respectively. Remarkably, the sharp peak at 2180 cm⁻¹ (not observed in the precursor spectrum) represented the presence of terminal cyano (—C—N) or (—N═C═N—) groups in MCN-TZL. Without wishing to be bound by theory, it is believed that these cyano groups evolved during the rearrangement or cross linking of the triazole rings accompanied with the elimination of NH₃. The presence of triazole ring mode vibrations were realized from the absorption signal at 809 cm⁻¹ while the signals at low wave-numbers (ω-NH groups) were absent due to elimination of NH₃ molecules during pyrolysis. The stability of the triazole nucleus even at high temperatures allowed the attached 3-amino group of 3-AT to participate in cross linking. In addition, C—H and C═O bonding were not present due to the absence of signals at 2870 and 1700 cm⁻¹.

From the XPS, FTIR, and UV-Vis analysis, it was determined that the MCN-TZL material was mainly composed of extensive conjugation of sp² hybridized C and N atoms along with small amount of (—C≡N) terminating groups. The presence of clusters (strong R conjugation) with or without lone pair states at π band produced a broader tail (bathochromic/red shift) in the absorption spectrum and the band gap decreased to 2.20 eV. Without wishing to be bound by theory, it is believed that networks composed of conjugated heterocyclic ring systems can have absorptions due to π-π* and n-π* electronic transition. The weak peak around 250 nm in the UV-Vis diffuse reflectance data was assigned to n-π* transitions in aromatic C—N heterocycles and main peak in the range 400-550 nm with broader tail exhibited strong π-π* electronic transitions. However, the decreasing band gap was well balanced by the presence of (—C═N) species which disrupted the aromatic ring structure of the graphitic material and the band gap was not further decreased.

### 6. Photoluminescence

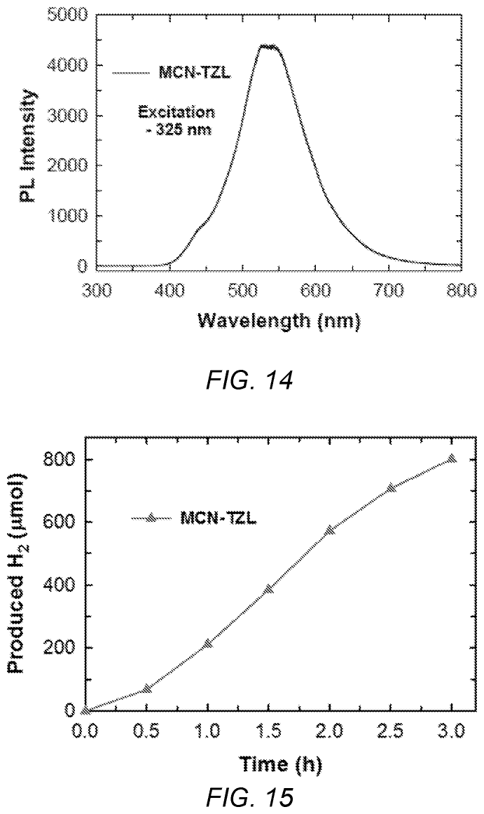

Photoluminescence of the MCN-TZL sample was in air by using the 325 nm line of a He—Cd laser as the excitation source. The sample surface was illuminated with a laser light with a spot diameter of ca. 0.5 mm. A spectrograph equipped with a CCD camera cooled with liquid nitrogen was used to analyze the PL emitted from the sample. All the spectra were corrected for the energy-dependent sensitivity of the detection system. The intensities were normalized with the excitation power density of each spectrum. FIG. 14 shows room temperature photoluminescence (PL) spectrum of MCN-TZL. As determined from this data, highly intense emission was observed at 540 nm (excitation 325 nm). As explained in the UV-Vis absorption section, the MCN-TZL material was a highly conjugated structure made up of sp² hybridized C and N atoms. It has been reported that PL emission strongly depends on the size of the sp² clusters in sp³ bonded matrix and π-π* as well as n-π* transitions. It has been reported that supramolecular or linear extension of π conjugated system leads can lead to strong luminescence properties. The strong luminescence at lower energy side (550 nm) observed for MCN-TZL was attributed to the photonic transitions in strong π electron density areas for example, between π-π* states and lone pair valence and σ* bands. In addition, photoluminescence properties strongly depend on the molecular structure and molecular assembly. FIG. 14 shows a small hump at ca. 445 nm in the photoluminescence spectrum. Although small, this high energy contribution in the PL spectrum was attributed to the presence of nitrile (—C≡N) or diimide (—N═C═N—) surface structures as observed by FT-IR. It has been reported that non-porous graphitic carbon nitride prepared from the pyrolysis of melamine showed poor absorption (235-250 nm) and weak luminescence (435 nm). Noteworthy, CNx materials derived from the thermal decomposition of triazido-s-heptazine precursor did not show any photoluminescence properties and luminescence was quenched by increased concentrations of defects in the prepared CNx structure. As the energies at the maxima of the PL signal (550 nm) in FIG. 14 were close to the optical gap E_{Tauc} (2.2 eV), any correlation between the PL process and electronic trap or defect states centered in the middle of the band gap was ruled out. The considerable strong intensity of the present material indicated that the MCN-TZL are useful light emitting materials. These results are particularly relevant to the development of next generation emitting devices for optoelectronic and sensing applications where stable room-temperature emission and operation in harsh environments are required.

### Example 5

### Photocatalytic H₂ Evolution Activity of MCN-TZL

Water splitting of the MCN-TZL was performed. Reactions were carried out in a Pyrex® (Corning, Inc., USA) top-irradiation reaction vessel connected to a glass closed gas circulation system. H₂ production was performed by dispersing 100 mg well ground catalyst powder in an aqueous solution (100 mL) containing triethanolamine (10 vol. %) as sacrificial electron donor. Pt was photodeposited on the catalysts using H₂PtCl₆ dissolved in the reactant solution. The reactant solution was evacuated several times to remove air completely prior to irradiation under a 300 W Xe lamp and a water-cooling filter. The wavelength of the incident light was controlled by using an appropriate long pass cut-off filter. The temperature of the reactant solution was maintained at room temperature by a flow of cooling water during the reaction. The distance between the light source and the reactor surface was 1.5 cm. The evolved gases were analyzed by gas chromatography equipped with a thermal conductive detector

FIG. 15 shows the time course of hydrogen evolution obtained over MCN-TZL sample under visible light irradiation. The production of hydrogen increased steadily with prolonged time of light irradiation (0-3 hours). After 3 hours of irradiation, a total of 801 mol H₂ gas (H₂ evolution rate, ca. 267 μmol·h⁻¹) was produced. This was significantly large as compared with reported graphitic carbon nitrides (<100 μmol·h⁻¹). The higher activity of Pt loaded MCN-TZL of the present invention was attributed to the reduced band gap of the material (E_{g}=2.20 eV), which allowed the MCN-TZL material to absorb large portion of the visible light from the solar spectrum. A band gap of 2.20 eV was large enough to overcome the thermodynamic barrier of the water splitting reaction. Second the higher activity of MCN-TZL was attributed to the high surface area (296.7 m²g⁻¹) and 3D mesoporous structure of the catalyst, which permits easy access to the photocatalytic active sites. The active sites were the interface between Pt co-catalyst and MCN-TZL framework. Platinum functioned as an efficient H₂ evolution promoter due to small overpotential. Thus, Pt loaded on the MCN-TZL effectively captured photogenerated electrons in the conduction band of MCN-TZL and host active site for H₂ evolution. The probability of radiative recombination of photoinduced charge carriers was depressed significantly by using the Pt co-catalyst.

### Example 5

### (Quartz Crystal Microbalance (QCM) Study)

### General Procedures

Quartz Crystal Microbalance. A QCM technique was used for detection of mass change during the assembly process. In order to act as electrodes, the QCM resonators (USI System, Japan) used were coated by vapor deposition with silver on both surfaces. The resonance frequency was 9 MHz (AT-cut) and frequency decreased (−ΔF) proportionally with increase in mass (Δm) according to the Sauerbrey equation. Using intrinsic parameters for AT cut quartz plate and electrode area, the equation Δm (Hz)=0.95×(−ΔF) (ng) holds. The frequency of the resonators was measured for adsorption step and the frequency was recorded when it became stable. The QCM frequency in air was stable within ±2 Hz during 1 hour. All experiments were carried out in an air-conditioned room at 25° C.

QCM Vapor Adsorption. For measuring the vapor adsorption, solvents (10 ml) in the 15 ml petri dish was kept into the trough in an QCM instrument in an air-conditioned room at 25° C. The QCM resonators with samples were then fixed in the QCM instrument. The QCM instrument was covered with a full side cover to prevent the vapor from leaking during the in situ adsorption measurement.

### Sensor Evaluation

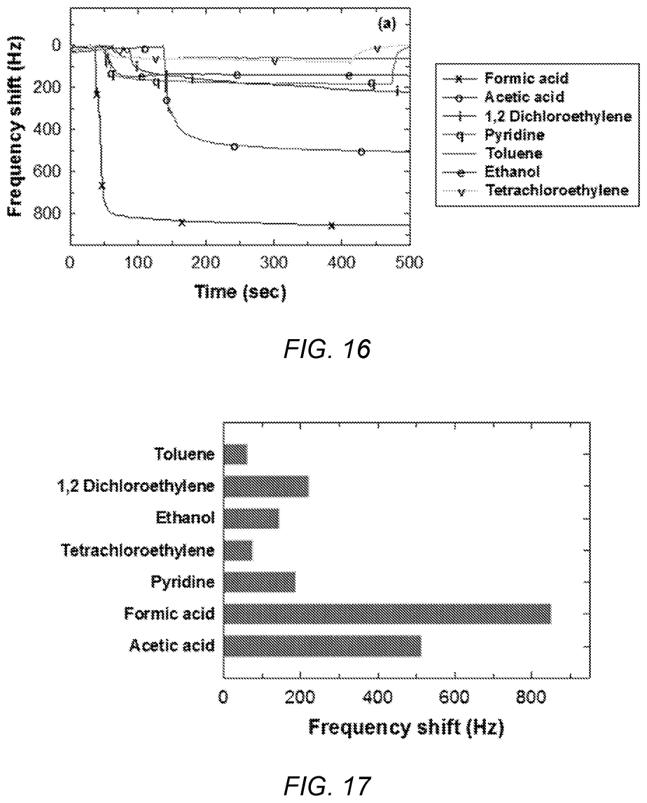

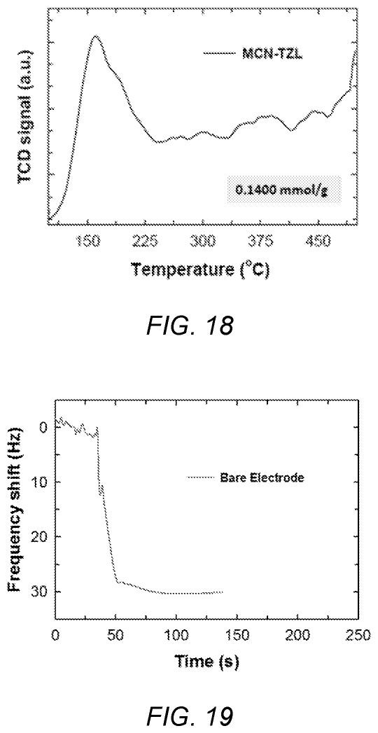

The MCN-TZL material was analyzed as a sensor. FIG. 16 displays the typical response patterns obtained from the QCM sensor when the MCN-TZL sample is exposed to volatile organic compounds such as aromatic hydrocarbons (toluene, aniline), acidic solvent (acetic acid, formic acid), alkaline solvent (pyridine) and hazardous ground water pollutants (1,2 dichloroethylene and tetrachloroethylene). In order to realize the magnitude of frequency shifts for different analytes, total frequency shift is depicted in bar graph (FIG. 17). Different amplitudes of frequency shift were observed for different guest molecules. However, frequency shift for the toxic formic acid and acetic acid molecules was larger compared with other guests despite their very similar vapor pressures and molecular weights, indicating the higher sensitivity and selectivity of MCN-TZL sample towards acidic solvent. First rapid frequency drop can be attributed to the adsorption of guest molecules on the outer surface of the film and the subsequent frequency drop was due to the penetration (diffusion) of the vapor molecules from gas phase inside the porous channels of the sample. Such a large selective adsorption of toxic acetic acid molecules was believed to due to the following two rationales. First, from the TPD spectrum of MCN-TZL (FIG. 18) it was determined that the presence of increased numbers of active Lewis basic sites (—NH₂, —NH) on the surface of the catalyst. This led to the sorption of acidic molecules through acid-base interaction. Second, the three dimensional structure with large specific surface area is believed to offers widely opened spaces and access to an increased number of basic sites for the guest-host interaction. To bear out the adsorption contribution from the gold electrode coated on the quartz crystal, measurements were done on the bare QCM electrode (FIG. 19) for acetic acid molecules. Bare QCM electrode showed very little frequency shift (30 Hz) which proved that the huge shift observed for the MCN-TZL coated electrode was attributed solely to the material performance. Apart from the acid base interaction and textural parameters contribution, dipole interactions and hydrogen bonding can also be one of the crucial factors in sensing studies. Noteworthy, MCN-TZL showed considerable response for ground water pollutant, 1,2 dichloroethylene. The response was attributed to the interaction of polar 1,2 dichloroethylene molecules with C—N dipoles due to the stronger dipole forces. The frequency response was correlated to the magnitude of the dipole moments of screened compounds. Dipole moment in descending order are: pyridine (2.21 D)>1,2 dichloro-ethylene (1.90 D)>ethanol (1.69 D)>tetrachloroethylene (1.2 D)>toluene (0.37 D). The magnitude of frequency shift was determined to be: 1,2 dichloro-ethylene (220)>pyridine (185 Hz)>ethanol (143 Hz)>tetrachloroethylene (75 Hz)>toluene (62 Hz). Due to a basic nature, pyridine showed less sorption even though its dipole moment was higher than that of 1,2 dichloroethylene. However, as shown in the sensing measurements, the effect of acid-base interaction was much higher as compared with the polar interactions between analyte molecules and C—N dipoles of the MCN-TZL. Thus, acidic compounds, which have lower dipole moments (acetic acid-1.70 D and formic acid-1.42 D) showed higher selectivity and sensitivity than that of other tested compounds.

### Example 6

### Temperature Programmed Desorption of CO₂

In order to measure the number and strength of basic sites on the MCN-TZL, as evidenced from XPS and FT-IR and speculated in QCM measurement, Temperature Programmed Desorption (TPD) of carbon dioxide (CO₂) was performed on the MCN-TZL sample using a Micromeritics® AutoChem II 2920 (Micromeritics®, USA) fully automated chemisorption analyzer equipped with gold-plated filament. The system consists of adjustable oven to heat the sample and gas mixture supplier for different gases. The measurements can be carried out from ambient to 1100° C. temperature range. In the present study, high purity carbon dioxide gas was used as a probe gas. About 80 mg of the samples were evacuated for 3 hrs at 250° C. under vacuum. Then samples were cooled to room temperature followed by CO₂ adsorption for 30 min. The physisorbed CO₂ was removed by heating the sample to 120° C. for 2 hrs. Desorption of chemisorbed CO₂ was performed in the temperature range of 120-500° C. with a rate of 5° C./min using a TCD detector.

FIG. 18 shows the profile for CO₂ desorption from MCN-TZL material. Broad desorption peak centered at 163° C. was observed. Since the area under the peak is proportional to the density of CO₂ molecules adsorbed (i.e., proportional to the surface coverage) on the surface of the sample, it was evident that the sample adsorbed a moderate amount of CO₂ molecules (0.140 mmol/g) on its surface. Such a considerable adsorption of CO₂ molecules was attributed to the existence of more number of basic sites (—NH₂, —NH— groups) on the surface of the sample. This acid-base interaction play important role in the chemisorption of the acidic molecules on the basic catalyst surface. Another factor responsible for the CO₂ adsorption was the high specific surface area and three-dimensional structure of the MCN-TZL, which provided enough exposure to more number of active basic sites.

In summary, novel mesoporous carbon nitrides are described with unique semiconducting properties from self-condensation reaction of 3-amino-1,2,4-triazole via nanocasting approach using KIT-6 silica template. The materials were characterized by various techniques such as XRD, nitrogen adsorption, TEM, EDX, EELS, XPS, FT-IR, UV-Vis spectroscopy, photoluminescence and elemental analysis. All of the characterization data suggest the presence of a highly basic, well ordered, 3D mesoporous carbon nitride with graphitic pore walls and very high nitrogen content, which exhibit semiconducting properties. The precursor used in the synthesis is commonly available and nontoxic. The combination of these properties and their ease of formation can make MCN-TZL of the present invention suitable for applications in absorption of bulky molecules, catalysis, light emitting devices, photocatalytic water splitting, as a storage material, sensing device, solar cells, etc.

1. A mesoporous carbon nitride (CN) material having a three dimensional matrix, an atomic nitrogen to carbon (N:C) ratio of 1.4 to 1.7, and a band gap of 1.8 to 3 eV.

2. The mesoporous CN material of claim 1, wherein the band gap is 2.2 eV.

3. The mesoporous CN material of claim 1, wherein the material is yellow.

4. The mesoporous CN material of claim 1, wherein the material has a BET surface area of 250 to 325 m²/g, a pore volume of 0.2 to 0.6 cm³g⁻¹, a pore size of 2 to 5 nm, or any combination thereof.

5. The mesoporous CN material of claim 1, further comprising a co-catalyst.

6. The mesoporous CN material of claim 5, wherein the co-catalyst comprises titanium, nickel, palladium, platinum, rhodium, ruthenium, tungsten, molybdenum, gold, silver, or copper, or combinations thereof.

7. The mesoporous material of claim 6, wherein the co-catalyst is platinum metal.

8-9. (canceled)

10. A photocatalytic process for producing hydrogen gas (H₂) from water, the process comprising:
(a) contacting the mesoporous material of claim 1 with water to form a reactant mixture; and
(b) exposing the reactant mixture to light to form hydrogen gas from the water.

11. A C₁₋₂ hydrocarbon acid sensor comprising the mesoporous material of claim 1.

12. The sensor of claim 11, wherein the C₁₋₂ hydrocarbon acid comprises formic acid, acetic acid, or both.

13. A method of producing a nitrogen rich mesoporous material of claim 1, the method comprising:
(a) obtaining an template reactant mixture comprising a calcined mesoporous KIT-6 template having a porosity and a protonated 3-amino-1,2,4-triazole;
(b) heating the template reactant mixture to form a CN/KIT-6 composite;
(c) heat treating the CN/KIT-6 composite to a temperature of 450° C. to 550° C. to form a cubic mesoporous carbon nitride material/KIT-6 complex; and
(d) removing the KIT-6 template from the cubic mesoporous carbon nitride material/KIT-6 complex.

14. The method of claim 13, wherein the heating of step (b) comprises:
heating to a first temperature is 90° C. to 110° C., preferably about 100° C. for 4 to 8 hours, preferably, 6 hours; and
increasing the temperature to 150° C. to 170° C., preferably about 160° C. for 4 to 8 hours, preferably 6 hours.

15. The method of claim 13, wherein heat treating temperature is about 500° C.

16. The method of claim 13, wherein the CN/KIT-6 composite is heated under an inert gas atmosphere.

17. The method of claim 16, wherein the inert gas is argon.

18. The method of claim 13, wherein obtaining the template reactant mixture comprises adding calcined KIT-6X to an aqueous solution of 3-amino-1,2,4-triazole and hydrochloric acid.

19. The method of claim 13, further comprising
producing the KIT-6 template by:
obtaining a polymerization solution comprising amphiphilic triblock copolymer and tetraethyl orthosilicate (TEOS);
reacting the polymerization mixture at about 100 to 200° C., preferably 150° C. to form a KIT-6 template having interpenetrating cylindrical pores;
drying the KIT-6 template at 90° C. to 110° C., preferably; and
calcining the dried KIT-6 template in air at 500 to 600° C., preferably 540° C. to form the calcined KIT-6 template.

20. (canceled)
